(19) United States
(12) Patent Application Publication
(10) Pub. No.: US 2010/0279329 A1
(43) Pub. Date: Nov. 4, 2010

(54) METHOD FOR THE STABILISATION OF PURIFIED P-GLYCOPROTEIN

(75) Inventors: Adam Heikal, Dunedin (NZ); Marcus Allen, East Sussex (GB); Richard Callaghan, Oxfordshire (GB); Andrew William Lloyd, East Sussex (GB)

Correspondence Address: VOLPE AND KOENIG, P.C., UNITED PLAZA, 30 SOUTH 17TH STREET, PHILADELPHIA, PA, 19103, US

(73) Assignee: UNIVERSITY OF BRIGHTON, East Sussex (GB)

(21) Appl. No.: 12/676,288

(22) PCT Filed: Sep. 3, 2008
(86) PCT No.: PCT/GB08/02979

§ 371 Date: Jun. 22, 2010

(30) Foreign Application Priority Data
Sep. 4, 2007 (GB) 0717166.3

(51) Int. Cl. C12Q 1/34; C12N 9/14

(52) U.S. Cl. 435/18; 435/195

(57) ABSTRACT

There is provided a method for producing purified P-glycoprotein (P-gp) in a stabilised form such that, after being reconstituted into proteoliposomes, it can be freeze dried and stored for prolonged periods of time without loss of biological activity The method comprises the steps of: i) solubilising a mixture of P-gp and other proteins obtained from cell membranes; ii) removing insoluble material by centrifugation; iii) placing the mixture of P-gp and other proteins in a purification column; and iv) running through the purification column at least one wash buffer so as to remove unwanted proteins, and then at least one elution buffer so as to recover the P-gp; wherein one or more of the buffers contains a disaccharide. Trehalose is a particularly preferred disaccharide.

This invention relates to a method for producing purified P-glycoprotein (P-gp) in a stabilised form such that, after being reconstituted into proteoliposomes, it can be freeze dried and stored for prolonged periods of time without loss of biological activity.

The membrane protein P-glycoprotein (P-gp), also known as ABCB1, is a clinically relevant multi-drug transporter or efflux pump. In normal physiology it is expressed in the membranes of cells in areas of the body which are either exposed to large numbers of xenotoxins, for example the lumen of the gut, the kidneys, etc, or areas particularly sensitive to such agents, for example the blood-brain barrier, the testis, etc. P-gp has a very unusually large range of substrates given transporters are typically restricted to one or two substrate molecules or types. The label of “multi-drug transporter” derives from the fact that, using ATP as an energy source, P-gp is capable of effluxing an enormous range of drugs from cells. These drugs often bear very little similarity to each other, either structurally or functionally, apart from them all being hydrophobic.

Spanning the plasma membrane of a cell, P-gp prevents drugs from building up inside the cell by removing them from the lipid bilayer that forms the cell membrane and transporting them back to the extra-cellular space. All new drugs must be screened against P-gp to determine whether they will enter sensitive areas of the body. P-gp mediated drug exclusion from cells is also a major feature of the multi-drug resistance (MDR) phenotype in some cancers. MDR presents a significant obstacle in the effective chemotherapy of some tumours. Therefore a screening system incorporating P-gp would be of value to both the wider pharmaceutical industry and the oncology community.

High throughput screens (HTS) are in an area of technology which is increasingly becoming of interest to both large pharmaceutical companies and academic research. The very nature of HTS requires them to be able to be run quickly, easily and at a time and place convenient to the user. Stability of a screen and its component parts is therefore of importance.

Membrane proteins are difficult to work with due to the fact that they may be more sensitive to their immediate environment than proteins which exist in solution. This sensitivity often leads to a rapid loss of biological activity. Functioning protein is central to a HTS of this nature. As such, the stabilisation of a membrane protein against factors such as temperature and long storage periods would be a great advantage. Moreover, whilst some biological molecules have been successfully protected in this manner for some time, functioning proteoliposomes have not.

Freeze drying or lyophilisation is the removal of water from a frozen system first by sublimation and then by desorption. It is a technique that has been used for some time and with a great deal of success over a wide range of applications. However, the removal of water from a biological system, whilst precluding certain degredative reactions from taking place, can itself be harmful. As such, in some cases the use of lyoprotectants is called for to help protect the structure and function of biological molecules. Although different classes of molecules are used, one set of molecules which are particularly good at protecting freeze dried systems is the disaccharides. These are added as excipients.

P-gp is purified by metal affinity chromatography. It is first solubilised from cell membranes, insoluble material is then removed via centrifugation and contaminating proteins are washed off a purification column before P-gp is eluted. The eluting buffer contains lipids and cholesterol but also detergent. In order for P-gp to be incorporated into the lipid environment it requires, the detergent must be removed. This is achieved by incubation with biobeads which slowly removes detergent and at the same time forces the formation of liposomes. As the detergent is removed, the hydrophobic lipids associate with each other and the hydrophobic portions of P-gp due to thermodynamic pressures from the increasingly aqueous solution. This produces a proteoliposome where the P-gp is embedded into the lipid bilayer much like it is in a P-gp expressing cell. This is the method described in detail by Taylor et al. (2001) Br. J. Pharmacol. 134, 1609-1618.

As will be apparent from the foregoing, P-gp presents a major challenge to the effective chemotherapy of some cancers and affects the bioavailability of a wide range of drugs. As such, new drugs are tested against P-gp in a variety of manners. In cancer research P-gp and P-gp proteoliposomes are used to test and develop new chemotherapeutic agents and/or P-pg inhibitors. Unfortunately, in its purified form, reconstituted into proteoliposomes, P-gp only retains ATPase activity over time when stored at −80° C. Lyophilisation has been used to successfully preserve biological molecules; however, to date no freeze drying of a proteoliposome system has been reported. There is therefore a need for an improved method for the purification and stabilisation of P-gp proteoliposomes.

According to the present invention there is provided a method for producing purified P-glycoprotein (P-gp) which comprises:
i) solubilising a mixture of P-gp and other proteins obtained from cell membranes;
ii) removing insoluble material by centrifugation;
iii) placing the mixture of P-gp and other proteins in a purification column; and
iv) running through the purification column at least one wash buffer so as to remove unwanted proteins, and then at least one elution buffer so as to recover the P-gp; wherein one or more of the buffers contains a disaccharide. Trehalose is a particularly preferred disaccharide for use in this invention. Other disaccharides, such as maltose, or a mixture of disaccharides, could also be used.

The present invention also provides purified P-gp prepared by the aforementioned method. There is still further provided P-gp proteoliposomes reconstituted from the purified P-gp prepared according to the method of this invention. There is also provided a screening system for drugs which comprises the use of P-gp or P-gp proteoliposomes produced by the method of this invention.

The method of the present invention makes possible the purification and stabilisation of P-gp proteoliposomes via lyophilisation. P-gp produced in this way retains up to 80% ATPase activity after lyophilisation and storage at 4° C. and 20° C. for up to 60 days, a significant improvement on un-lyophilised proteoliposomes.

According to this invention, P-gp is purified by a modified version of the conventional method described by Taylor et al. and summarised above. The new and non-obvious alteration to this method which allows the stabilisation of P-gp proteoliposomes is as follows. During washing of the column at the point of lowering the pH from 8 to 6.8 the constitution of the buffer was changed. The conventional wash and elution buffer contains 20 mM Tris, 150 mM NaCl, 1.5 mM MgCl₂, 1.25% (w/v) OG, 0.1% (w/v) lipids/cholesterol mix and 20% (v/v) glycerol. However, in the method of this invention the buffer constitution was changed to 20 mM Iris, 150 mM NaCl, 1.5 mM MgCl₂, 1.25% (w/v) OG, 0.1% (w/v) lipids/cholesterol mix and 20% (w/v) disaccharide. It will be appreciated that the proportion of the disaccharide used may be varied, but is typically from 10-30%, preferably from 15-25% and most preferably about 20% (w/v).

Thus, by altering the composition of the final buffer used to elute P-gp from the purification column it has surprisingly been found possible to freeze dry P-gp containing proteoliposomes and, on re-hydration, retain biological activity in the protein. Traditional protein purification buffers contain a high proportion of glycerol, an excellent cryoprotectant. However, this precludes freeze drying and affords no real protection against drying stresses. By using a buffer that contains a disaccharide, most preferably, trehalose, during the final stages of the purification, it has been possible to freeze dry the proteoliposomes. It may be possible to use a disaccharide containing buffer throughout the purification, rather than it only being used for elution.

While the production of proteoliposomes and freeze drying of both liposomes and proteins have been known for some time, the successful stabilisation of proteoliposomes by freeze drying is a new and highly advantageous development achieved by the present invention. The result is a system where membrane proteins are incorporated into a lipid bilayer as part of a liposome which is then protected from loss of biological activity via freeze drying. This is the basis for the development of a high throughput screen for new drugs as the system can withstand a greater range of conditions for both handling and storage.

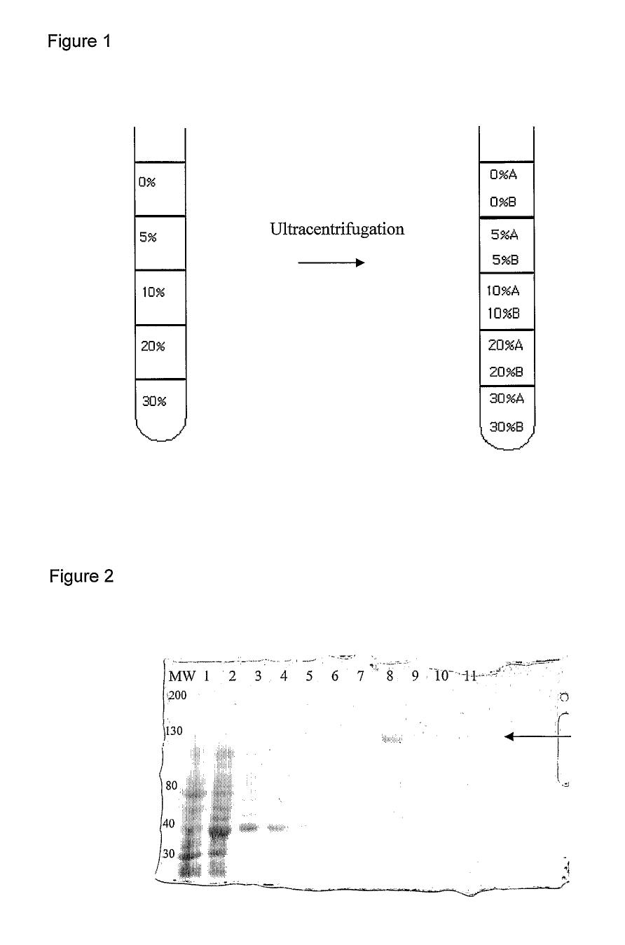

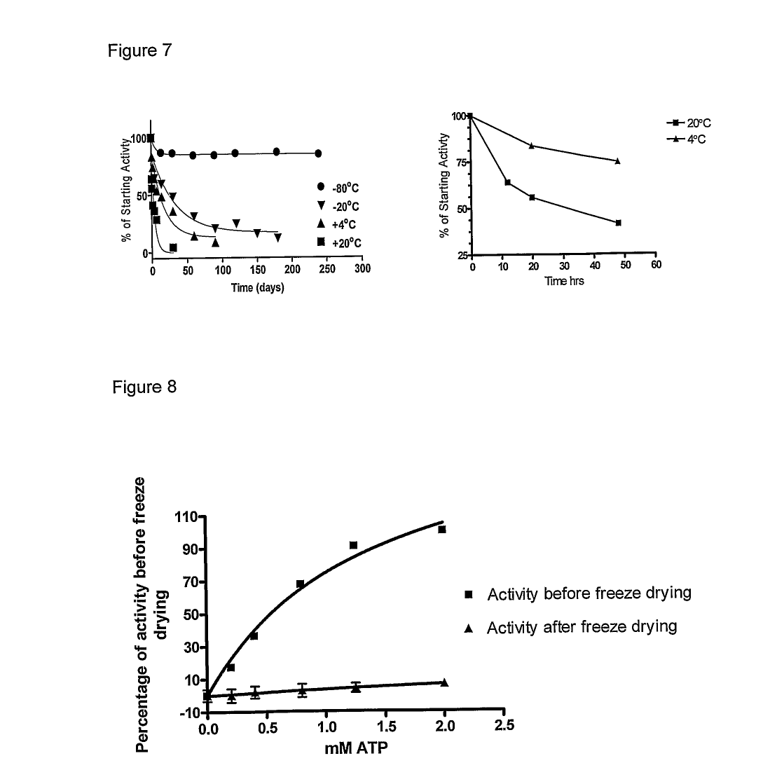

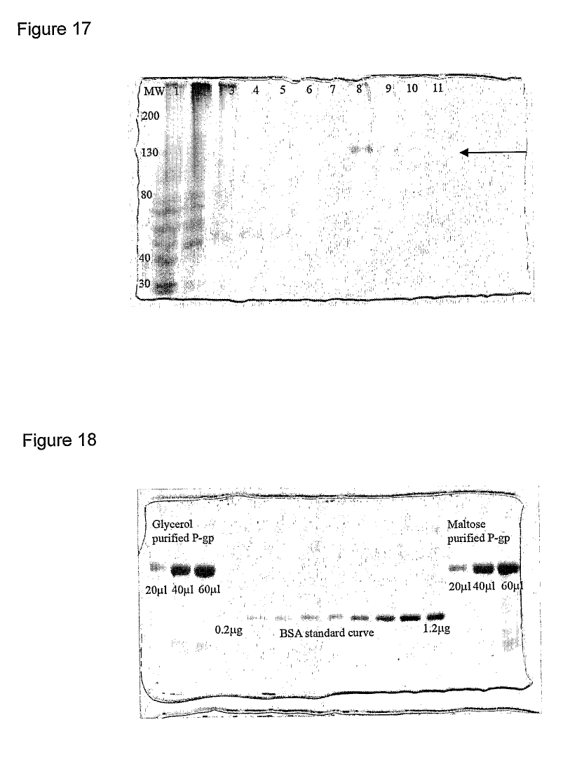

The present invention will now be described in more detail by reference to the following Examples and the accompanying Figures (FIGS. 1 to 6 relate to Example 1, FIGS. 7 to 16 relate to Example 2 and FIGS. 17 to 23 relate to Example 3):

FIG. 1: Diagram to represent setting up and subsequent harvesting of a sucrose density gradient. 400 μl layers were poured into an ultracentrifuge tube so as to avoiding mixing of different sucrose concentrations. This was then ultra-centrifuged at 150,000 g overnight. On completion 200 μl fractions were harvested as A fractions and B fractions.

FIG. 2: SDS-PAGE results from Example 1. Lane markings from left to right: MW=molecular weight markers; 1=unbound protein; 2=10 mM imidazole pH 8; 3=10 mM imidazole pH 8; 4=2 mM imidazole pH 8; 5=30 mM imidazole pH 8; 6=2 mM imidazole pH 6.8; 7=120 mM imidazole pH 6.8 flow through; lanes 8 to 11=120 mM imidazole pH 6.8 with 2 minute incubation. Arrow indicates position of P-gp.

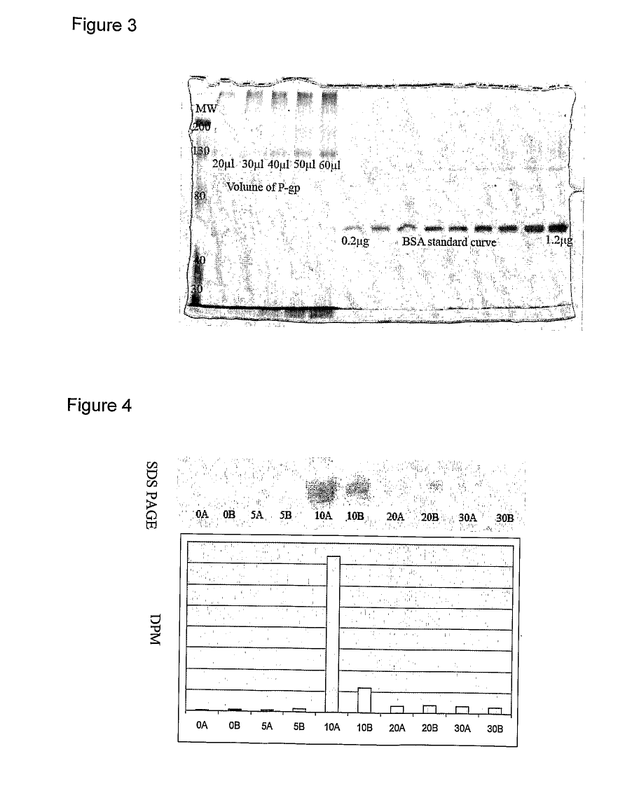

FIG. 3: Example of an SDS-PAGE stained with Coomassie Blue. Volumes of purified P-gp were run next to known amounts of BSA as a standard curve. P-gp concentration was calculated using densitometric analysis of bands shown on the gel.

FIG. 4: Upper panel shows position of P-gp as visualised by silver stained SDS-PAGE. The location of [³H]-phosphatidylcholine is shown as a bar chart of DPM as determined by liquid scintillation counting. Protein and lipid are seen to be mainly in fractions 10A and 10B indicating a good reconstitution.

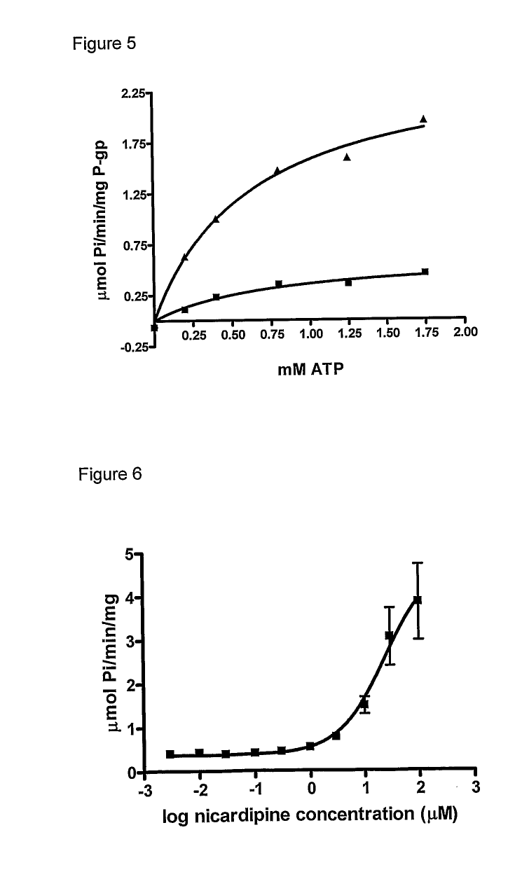

FIG. 5: Graph showing P-gp basal non-drug stimulated and drug stimulated (30 μM nicardipine) ATPase activity. Basal Vmax was 0.62 μmol/min/mg, nicardipine stimulated Vmax was 2.57 μmol/min/mg (n=4 independent purifications).

FIG. 6: Graph showing a dose response curve of drug stimulated ATPase activity against nicardipine. EC50 was determined by fitting the general dose response equation as 24.2 μM nicardipine. Error bars show SEM from 4 independent purifications.

FIG. 7: Graphs showing loss of ATPase activity of P-gp proteoliposomes over time when stored at different temperatures.

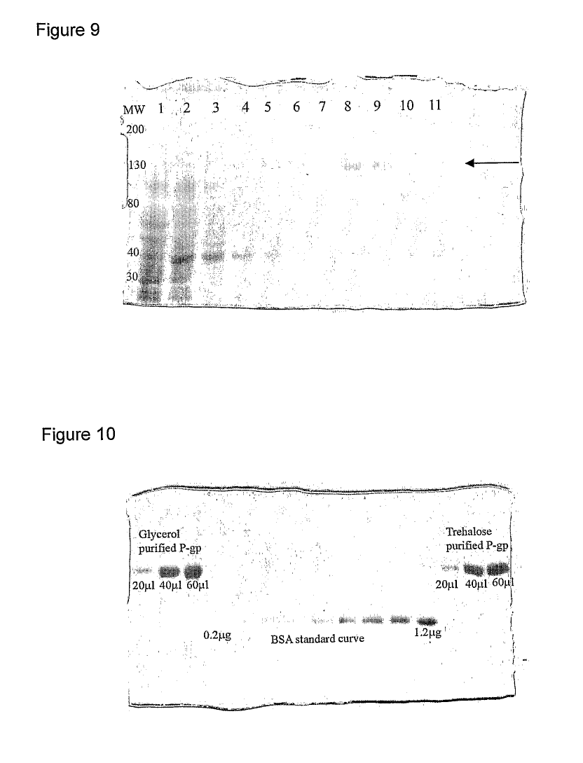

FIG. 8: Graph showing loss of nicardipine stimulated ATPase activity on freeze drying of conventionally purified P-gp proteoliposomes. No ATPase activity was recovered on rehydration and assay of P-gp proteoliposomes. Error bars are SEM n=4.

FIG. 9: SDS-PAGE results from Example 2. Lane markings from left to right: MW=molecular weight markers; 1=unbound protein pH 8; 2=10 mM imidazole pH 8; 3=10 mM imidazole pH 8; 4=20 mM imidazole pH; 5=30 mM imidazole pH 8; 6=2 mM imidazole pH; 7=120 mM imidazole flow through pH 6.8; lanes 8 to 11=120 mM imidazole with 2 minute incubation pH 6.8. Arrow indicates position of P-gp.

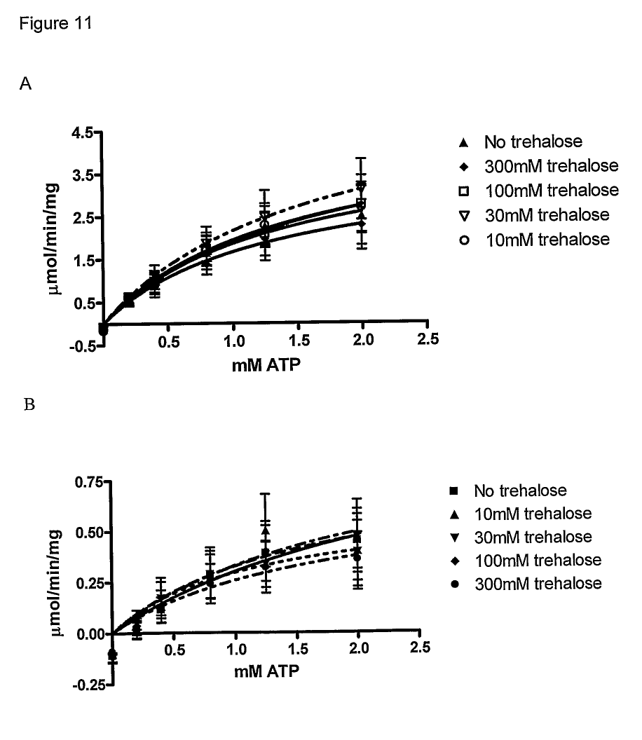

FIG. 10: Example of BSA gel with both glycerol purified P-gp and trehalose purified P-gp visualised by way of comparison. A BSA standard curve running from 0.2 μg-1.2 μg was used to determine the amount of purified P-gp via densitometric analysis.

FIG. 11: (A) Graph showing Vmax for nicardipine (30 μM) stimulated ATPase activity at 0 mM (filled triangles), 10 mM (open circles), 30 mM (open inverted triangles), 100 mM (open squares) and 300 mM (filled diamonds) trehalose. Two way ANOVA shows no significant difference between Vmax in the presence of trehalose and without (p=0.6551). (B) Graph showing basal ATPase activity at 0 mM (filled squares), 10 mM (filled triangles), 30 mM (filled inverted triangles), 100 mM (filled diamonds) and 300 mM (filled circles) trehalose. Error bars show the Standard Error of the Mean (SEM) from 4 independent purifications in the presence of different concentrations of trehalose. Two way ANOVA shows no significant difference between Vmax in the presence of trehalose and without (p=0.9422).

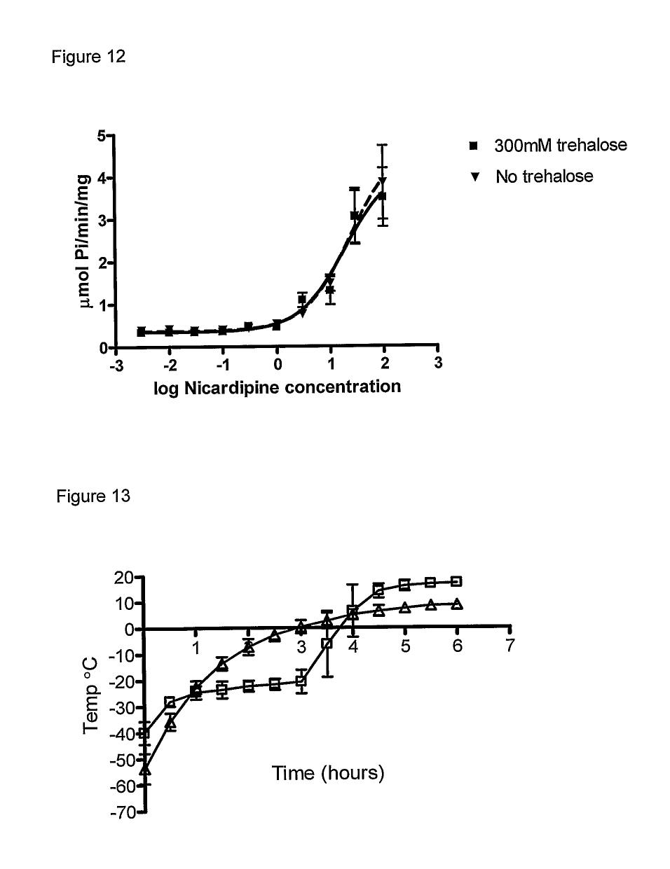

FIG. 12: Graph showing a dose response curve of drug stimulated ATPase activity against nicardipine. Dotted line and triangles show ATPase activity in absence of trehalose, solid line and squares are ATPase activity with 300 mM trehalose. EC50 was determined by fitting the general dose response equation as 19.2 μM nicardipine. Error bars show SEM from 4 independent purifications. Two way ANOVA shows no significant difference between the two curves (p=0.7549).

FIG. 13: Graph shows temperature change of freeze drying shelf and product over time. Temperature was measured using thermocouples embedded in the shelf and frozen into the product prior to lyophilisation. Open triangles represent shelf temperature and open squares the product. Error bars are the SEM of n=4. Chamber pressure was 0.011 mBar throughout.

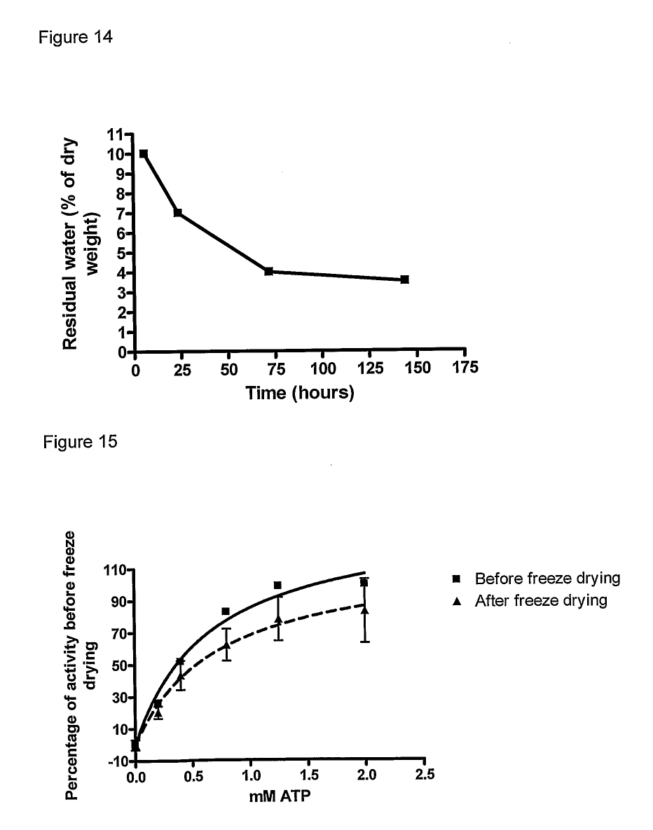

FIG. 14: Graph showing how the residual moisture of freeze dried product varies with time. Drying pressure was 0.011 mBar throughout. Increasing drying time from 72 hours to 144 hours only resulted in a further 0.5% reduction of residual moisture.

FIG. 15: Comparison of pre and post freeze dried P-gp proteoliposome ATPase activity. Samples were rehydrated immediately after freeze drying cycle was completed with room temperature double distilled water. 100% was deemed to be nicardipine stimulated Vmax prior to freeze drying. The solid line and squares represents pre freeze dried activity, dashed line and triangles represents post freeze dried activity.

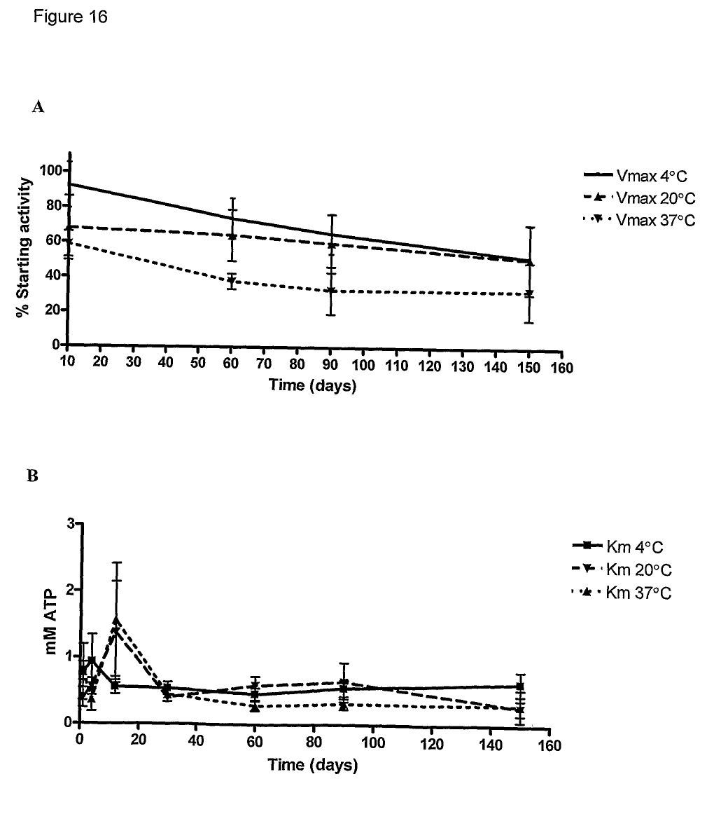

FIG. 16: (A) Nicardipine stimulated ATPase activity of freeze dried trehalose purified P-gp proteoliposomes over time. 100% was deemed to be nicardipine stimulated Vmax prior to freeze drying. Solid line and squares represents storage at 4° C., dash line and triangles 20° C. and dotted line and inverted triangles 37° C. Error bars are the SEM of n=4 independent purifications. (B) Graph showing Km of 30 μM nicardipine stimulated ATPase activity in freeze dried P-gp proteoliposomes over time. Solid line represents samples stored at 4° C., dashed line samples at 20° C. and dotted samples 37° C. Error bars are SEM of n=4 independent purifications.

FIG. 17: SDS-PAGE results from Example 3. Lane markings from left to right: MW=molecular weight markers; 1=unbound protein pH 8; 2=10 mM imidazole pH 8; 3=10 mM imidazole pH 8; 4=20 mM imidazole pH 8; 5=30 mM imidazole pH 8; 6=2 mM imidazole pH 8; 7=120 mM imidazole flow through pH 6.8; lanes 8 to 11=120 mM imidazole with 2 minute incubation pH 6.8. Arrow indicates position of P-gp.

FIG. 18: Example of BSA gel with both glycerol purified P-gp and maltose purified P-gp visualised by way of comparison. A BSA standard curve running from 0.2 μg-1.2 μg was used to determine the amount of purified P-gp via densitometric analysis.

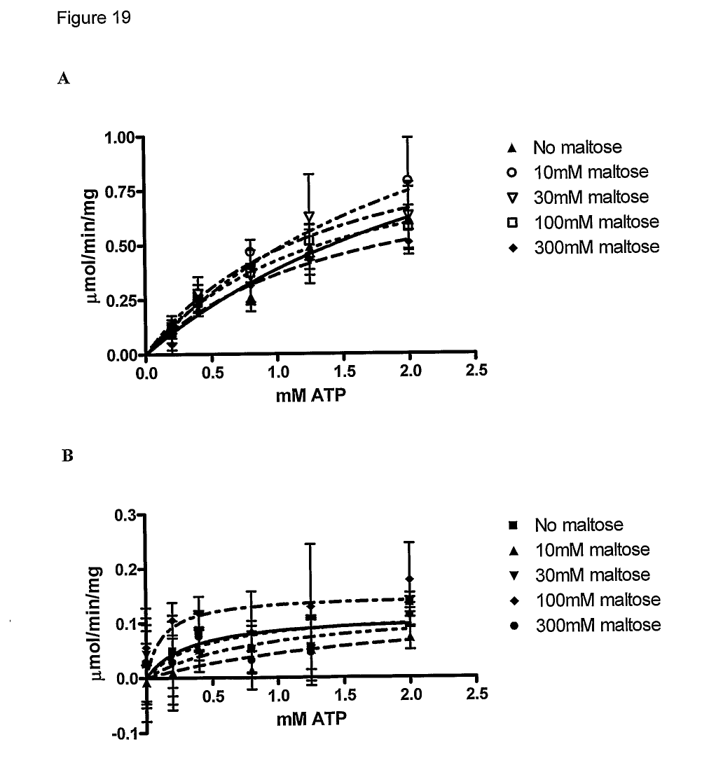

FIG. 19: (A) Graph showing Vmax for nicardipine (30 μM) stimulated ATPase activity at 0 mM (filled triangles), 10 mM (open circles), 30 mM (open inverted triangles), 100 mM (open squares) and 300 mM (filled diamonds) maltose. Two way ANOVA shows no significant difference between Vmax in the presence of maltose and without (p=0.4215). (B) Graph showing basal ATPase activity at 0 mM (filled squares), 10 mM (filled triangles), 30 mM (filled inverted triangles), 100 mM (filled diamonds) and 300 mM (filled circles) maltose. Error bars show the Standard Error of the Mean (SEM) from 4 independent purifications in the presence of different concentrations of maltose. Two way ANOVA shows no significant difference between Vmax in the presence of maltose and without (p=0.2002).

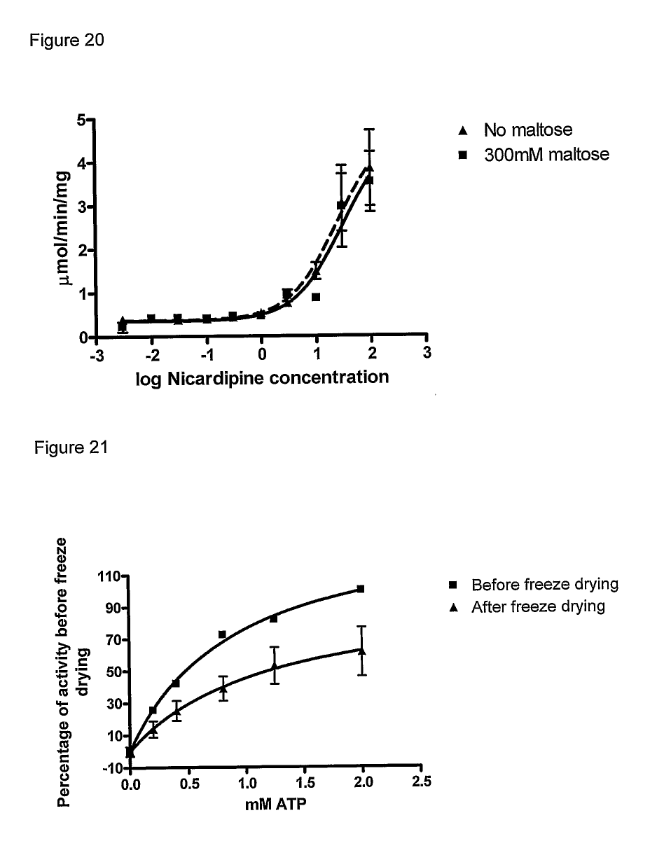

FIG. 20: Graph showing a dose response curve of drug stimulated ATPase activity against nicardipine. Dotted line and triangles show ATPase activity in absence of maltose, solid line and squares are ATPase activity with 300 mM maltose. EC50 was determined by fitting the general dose response equation as 30.02 μM nicardipine. Error bars show SEM from 4 independent purifications. Two way ANOVA shows no significant difference between the two curves (p=0.5249).

FIG. 21: Comparison of pre and post freeze dried P-gp proteoliposome ATPase activity. Samples were rehydrated immediately after freeze drying cycle was completed with room temperature double distilled water. 100% was deemed to be nicardipine stimulated Vmax prior to freeze drying. The solid line and squares represents pre freeze dried activity, dashed line and triangles represents post freeze dried activity.

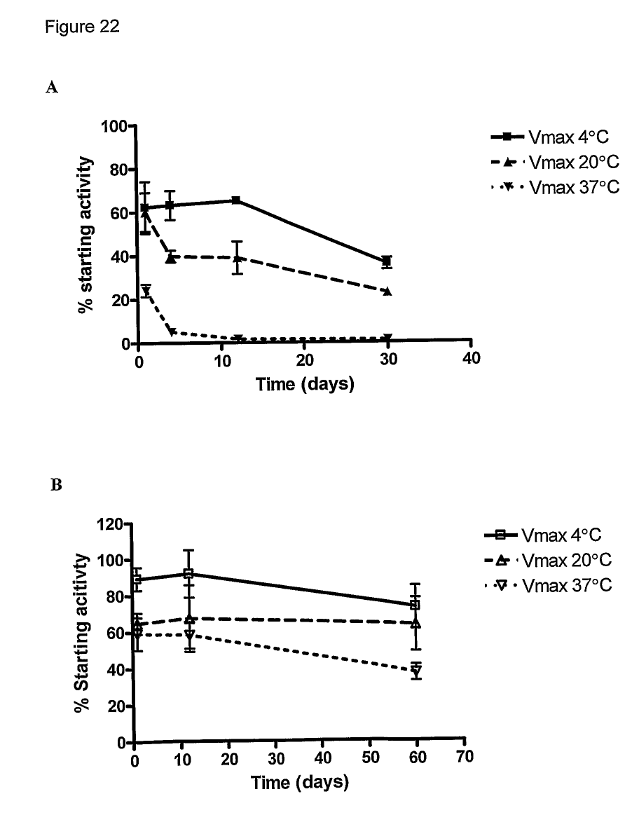

FIG. 22: (A) Nicardipine stimulated ATPase activity of maltose purified freeze dried P-gp proteoliposomes over time. 100% was deemed to be nicardipine stimulated Vmax prior to freeze drying. Solid line and squares represents storage at 4° C., dash line and triangles 20° C. and dotted line and inverted triangles 37° C. Error bars are the SEM of n=4 independent purifications. (B) Nicardipine stimulated ATPase activity of trehalose purified freeze dried P-gp proteoliposomes over time. 100% was deemed to be nicardipine stimulated Vmax prior to freeze drying. Solid line and open squares represents storage at 4° C., dash line and open triangles 20° C. and dotted line and open inverted triangles 37° C. Error bars are the SEM of n=4 independent purifications.

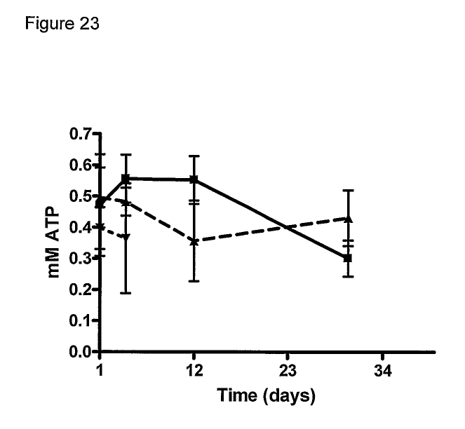

FIG. 23: Graph showing Km of 30 μM nicardipine stimulated ATPase activity in freeze dried maltose purified P-gp proteoliposomes over time. Solid line represents samples stored at 4° C., dashed line samples at 20° C. and dotted samples 37° C. Error bars are SEM of n=4 independent purifications.

### EXAMPLE 1

### Purification of P-gp

### 1.1 Materials.

Cholesterol, di-sodium adenosine tri-phosphate (Na₂ATP), nicardipine and buffer salts were obtained from Sigma (Poole, UK). DC detergent compatible protein assay kit and SM-2 Biobeads from BioRad (Hemel Hemstead, UK). Octyl-β-D-glucoside (OG), leupeptin hemisulfate, pestatin, benzamide HCl and Ni-NTA resin from Calbiochem (Nottingham, UK). E. coli total lipid extract from Avanti Polar Lipids (USA). [³H]-phosphatidylcholine (84 Ci/mmol) from Amersham Biosciences (UK). Silver staining kit for SDS-PAGE from ICN (Thame, UK). PageBlue™ gel staining system from Fermentas Life Sciences (USA).

### 1.2 Methods.

### 1.2.1 Cell Culture and Maintenance of Baculovirus.

The generation of recombinant baculovirus encoding (His₆)-tagged P-gp was carried out at the NDCLS, Oxford as described by Taylor et al. 2001. Briefly Spodoptera frugiperda (Sf900) were used to produce recombinant virus and Trichoplusia ni (High Five) cells were used for expression of (His₆)-tagged P-gp. Infection and culture of all cells was kindly performed by various members of the NDCLS.

### 1.2.2 Preparation of Crude Membranes.

High Five cells were pelleted via centrifugation (2000 g, 4° C., 10 minutes) and washed in ice cold PBS. The pellet was resuspended in membrane buffer 1 (0.01M Tris pH7.4, 0.25M sucrose and 0.2 mM CaCl₂) to give 5 times the original pellet volume. Protease inhibitors were added from ×100 stock to give 20 μM leupeptin, 1 mM benzamide and 2 μM pepstatin. The cells were then transferred to a nitrogen cavitation ‘bomb’ and subjected to 4 rounds of cavitation (˜1500 p.s.i., 15 minutes per round, 4° C.) to lyse the cells.

Non-lysed cells were then removed by centrifugation (2000 g, 4° C., 15 minutes) and the resulting supernatant was ultra centrifuged (100,000 g, 4° C., 45 minutes) to isolate membranes. The pellet from ultra centrifugation was then resuspended in a volume of membrane buffer 2 (as membrane buffer 1 but without CaCl₂) equal to the volume of the original cell pellet. Protease inhibitor was added to give ×2 final concentration. Aliquots of membrane preparation were stored at −80° C.

### 1.2.3 Confirmation of P-gp Expression.

After preparation of crude membranes from High Five cells, P-gp expression was confirmed by immunoblotting (Ronchi et al., 1989). Using the DC detergent compatible protein assay kit (BioRad, Hemel Hempstead) the total protein concentrations of a membrane preparation known to contain P-gp and a preparation thought to contain P-gp were determined. Samples containing 5, 10 and 20 μg of total membrane protein were run on 7.5% SDS-PAGE and Western blotted. The P-gp specific primary antibody, C219, and then anti-mouse Ig horseradish peroxidase linked antibody were used to visualise P-gp.

### 1.2.4 Solubilisation of Membrane Proteins for Chromatography.

E. coli lipids (phosphatidylethanolamine, phosphatidylglycerol and cardiolipin) and cholesterol were dissolved in chloroform:methanol (2:1) at 100 mg/ml and mixed to give E. coli:cholesterol (4:1). They were dried under a stream of nitrogen and then vacuum for at least 1 hour. By adding purification buffer 1 (20 mM Tris pH 6.8, 150 mM NaCl, 1.5 mM MgCl₂ and 20% (v/v) glycerol) with octyl-β-D-glucoside (OG) and repeated sonication and vortexing till clear the lipids and cholesterol were resuspended and incorporated into the buffer. A volume of crude membranes were diluted in purification buffer 1 (20 mM Tris pH 6.8, 150 mM NaCl, 1.5 mM MgCl₂ and 20% (v/v) glycerol) sufficient to give 50 mg total protein and was centrifuged (100,000 g, 4° C., 20 minutes). The pellet was then resuspended in purification buffer 1 supplemented with 2% (w/v) OG to solubilise membrane proteins and 0.4% (w/v) E. coli lipid and cholesterol mixture.

The resuspended membrane pellet was then incubated at 4° C. for 40 minutes to allow the detergent to solubilise the membrane proteins. Any remaining insoluble material was removed via further centrifugation (100,000 g, 4° C., 20 minutes), the supernatant containing soluble material.

### 1.2.5 Purification of P-gp.

The use of Ni-NTA to purify solubilised P-gp has been previously reported in detail (Taylor et al. 2001) and is described below. Ni-NTA resin was washed in 20 bed volumes (bv) of water and then 20 bv solubilisation buffer by gentle centrifugation. The solubilised protein was then added to the resin and the mixture was incubated with gentle rocking at 4° C. for at least 1 hour. The mixture was then added to an empty Econo-column and allowed to flow through, removing any unbound proteins. The column was then washed sequentially by 20 bv of a series of wash and elution buffers (see Table 1).

All buffers contained 20 mM Tris, 150 mM NaCl, 1.5 mM MgCl₂, 1.25% (w/v) OG and 0.1% (w/v) lipids/cholesterol mix, 20% (v/v) glycerol and varying amounts of imidazole to remove non-specifically bound proteins. The pH was also changed so that P-gp remained bound via its histidine tag during washing but was eluted in the final stages of purification.

TABLE 1
pH and imidazole concentrations of buffers used during
purification and elution.

| | | Imidazole |
|---|---|---|
| Buffer | pH | concentration |
| Wash 1 | 8.0 | 10 mM |
| Wash 2 | 8.0 | 10 mM |
| Wash 3 | 8.0 | 20 mM |
| Wash 4 | 8.0 | 30 mM |
| Wash 5 | 6.8 | 2 mM |
| Elution | 6.8 | 120 mM |

P-gp was eluted in 2 bv fractions of elution buffer after incubation on the column for 2 minutes. 3% of all washes and elutions were TCA precipitated (Bensadoun et al., 1976) and run on SDS-PAGE. The gel was either silver stained (ICN) or stained using PageBlue™ (Fermentas Life Sciences) to monitor purification and identify P-gp containing fractions.

1.2.6 Reconstitution of P-gp into Lipid Bilayer of Liposomes.

Elution fractions containing P-gp were pooled and SM-2 BioBeads were added at 225 mg BioBeads/ml P-gp elution to remove detergent and thereby reconstitute P-gp into proteoliposomes (Rigaud et al., 1988). BioBeads were prepared by washing for 5 minutes on a shaker in water, then 50:50 water:methanol, methanol and finally water 3 times. To assess reconstitution efficiency a sucrose density gradient was employed. 1 μCi [³H] phosphatidylcholine was added as a lipid tracer. This was then mixed by rotation for 2 hours at room temperature.

Reconstitution efficiency was not always assessed after purification. However, demonstration of ATPase activity was deemed to be sufficient to confirm reconstitution as P-gp exhibits virtually no ATPase activity when not inserted into a lipid bilayer (Callaghan et al., 1997).

### 1.2.7 Assessment of Reconstitution Efficiency by Sucrose Density Centrifugation.

A fraction of the reconstituted P-gp (200 μl) was mixed with an equal volume of 60% (w/v) sucrose/0.05% Triton X-100. 400 μl of 20%, 10%, 5% and 0% sucrose solutions were layered over the sample. This was then centrifuged (150,000 g, 4° C., 12 hours) and 200 μl of each layer collected. The upper 200 μl fraction of each layer was labelled ‘A’ and the lower ‘B’ e.g. 30% A and 30% B (see FIG. 1). From each fraction 50 μl was then used to determine the distribution of [³H]-phosphatidylcholine via liquid scintillation counting (Ready Protein scintillant, LS6500 scintillation counter, Beckman). The remaining 150 μl was TCA precipitated, electrophoresed on SDS-PAGE and visualised with either silver stain or PageBlue™ to monitor the position of the protein relative to the lipids. Reconstitution was deemed successful when protein and lipid had co-migrated to the same fraction of the density gradient. The purified, reconstituted P-gp was then stored at −80° C.

### 1.2.8 Determination of Protein Concentration.

To determine the protein concentration of purified P-gp volumes of P-gp were run on SDS-PAGE next to known amounts of BSA. This results in a standard curve of BSA running from 0.2 μg-1.2 μg. The gel was then stained with Coomassie Blue (30 minutes minimum) and de-stained with 10% (v/v) acetic acid, 5% methanol (v/v) overnight ready for densitometric analysis. This was carried out using Scion Image software (Scion Corporation, US) and the concentration of P-gp was calculated from the BSA standard curve.

### 1.2.9 Varying ATP Concentration Michaelis-Menten Kinetics.

P-gp displays ATPase activity leading to the hydrolysis of ATP to ADP and inorganic phosphate (Pi). A colorimetric assay (Chifflet et al., 1988) was used to determine ATP hydrolysis via release of inorganic phosphate. P-gp activity was expressed as μmol Pi/min/mg protein.

Reconstituted P-gp (0.3 μg) was added to various concentrations of ATP (0-2 mM) in the presence (drug stimulated activity) or absence (basal activity) of 30 μM nicardipine in a 96 well plate. Nicardipine was added from a 50 mM stock in DMSO and the amount of DMSO was maintained at less than 1% (v/v) to ensure protein viability.

A 0-20 nmol standard curve was set up using KH₂PO₄ as a source of Pi. Background Pi was measured using wells containing no protein or drug only ATP and ATPase buffer (50 mM Tris pH 7.4, 150 mM NH₄Cl, 5 mM MgSO₄, 0.02% (w/v) NaN₃).

The plate was incubated for 20 minutes at 37° C. and the reaction quenched by rapid addition of 40 μl 12% (w/v) SDS to all wells. 100 μl of a 1:1 mixture of 6% (w/v) ascorbate in 1M HCl:1% (w/v) ammonium molybdate (prepared after the reaction had been quenched due to its sensitivity to light) was added to all wells and the plate was incubated for 5 minutes at room temperature. Finally, 100 μl 2% (w/v) sodium citrate/sodium metaarsentie/2% (v/v) acetic acid was added and the plate incubated at 37° C. for 15 minutes. The plate was then allowed to cool and the absorbance measured at a wavelength of 750 nm in a plate reader (SpectraMax 250, Molecular Devices/Multiskan Ascent). The amount of Pi released was calculated from the Pi standard curve and the ATPase activity of P-gp calculated.

The amount of protein (mg) in 20 μl was calculated from the protein concentration determined by BSA standard curve (see 1.2.8) allowing ATPase activity to be determined as μmol/min/mg protein. The Michaelis-Menten equation (Equation 1) was fitted to a graph of ATPase activity using non-linear least squares fit (GraphPad Prism 4.0).

$$V = \frac{V max [ S ]}{Km + [ S ]}$$

Equation 1: The Michaelis-Menten equation, where
V=rate of hydrolysis expressed as μmol/min/mg protein,
Vₘₐₓ=maximum rate of hydrolysis,
[S]=concentration of ATP (M), and
Kₘ=Michaelis constant (M).

### 1.2.10 Varying Drug Concentration at Constant ATP Concentration.

The ATPase activity of reconstituted P-gp was measured over a range of 0-100 mM nicardipine in the presence of 2 mM ATP. The purpose of this was to assess the interaction between P-gp and nicardipine. The assay was set up, conducted and read in conditions identical to those described above. ATPase activity was plotted against nicardipine concentration and the general dose response curve was fitted using non-linear fit (GraphPad Prism 4.0). The equation for the general dose response curve is given below (Equation 2).

$$V = \frac{( V max - V min )}{V min + ( 1 + {10}^{( {log}_{10} EC 50 - [ D ] )} )}$$

Equation 2: The general dose response curve equation, where
V=rate of hydrolysis expressed as μmol/min/mg Pi,
Vmax=maximum rate of hydrolysis,
Vmin=minimum rate of hydrolysis,
[D]=concentration of drug, and
EC50=concentration of drug required to produce half maximum rate of hydrolysis.

### 1.3 Results and Analysis of Purification.

### 1.3.1 Monitoring Purification via SDS-PAGE.

3% (or ˜2 μg) fractions of washes and elutions were visualised on SDS-PAGE (see FIG. 2). As can be seen in lanes 2-7 non-specifically bound proteins were removed. P-gp however is only eluted when the histidine tag's binding affinity for the Ni-NTA is reduced via lowering of pH and the protein is displaced by competitive binding from the increased imidazole concentration, lanes 8 -11.

### 1.3.1 Determining Yield of P-gp.

From crude membranes containing 50 mg total membrane protein 60 μg (n=4, SEM 2.3) As determined from a BSA gel such as FIG. 3. P-gp could be purified using a glycerol buffer. Based on evidence from previous studies (not published) that OG solubilised 50% of P-gp present, it can be concluded that 0.6% starting membrane proteins are P-gp.

### 1.3.2 Assessment of Reconstitution Efficiency.

As can be seen from FIG. 4 the vast majority of protein, visualised on SDS-PAGE in fractions 10A and B, can be seen to correlate to the position of the [³H]-phosphatidylcholine, shown by bar chart. This indicates that P-gp has inserted into the bi-layer of the liposome forming a proteoliposome and that P-gp will be able to display ATPase activity.

### 1.3.3 ATPase Activity of Purified P-gp.

To ensure purified P-gp was functional the ATPase activity was measured both with and without drug stimulation. FIG. 5 is an example of P-gp eluted in a glycerol buffer exhibiting ATPase activity. In the presence of 30 μM nicardipine the Vmax was found to be 2.6 μmol Pi/min/mg P-gp (n=4, SEM 0.15) with a Km of 0.6 mM ATP (n=4, SEM 0.04). Basal Vmax was 0.5 μmol Pi/min/mg P-gp (n=4, SEM 0.06) and Km 0.4 mM ATP (n=4, SEM 0.07).

The ATPase activity of P-gp was also measured over a range of nicardipine concentrations. The EC50 of nicardipine on P-gp was found to be 24.2 μM (see FIG. 6).

### 1.4 Summary of Results.

As the results presented above show, P-gp could be isolated and purified from insect cell membranes and then successfully reconstituted into proteoliposomes where it displayed both basal and nicardipine stimulated ATPase activity.

High Five cells expressing P-gp were disrupted via several rounds of nitrogen cavitation and any remaining non-lysed cells were removed by further centrifugation. Isolation of membranes was achieved through ultra-centrifugation with the pellet being resuspended in buffer. These membranes could be stored at −80° C. for long periods of time if necessary. At this point P-gp expression could be confirmed by immunoblotting with the specific primary antibody, C219, and then anti-mouse Ig horseradish peroxidase linked antibody to visualise P-gp. As a membrane protein, purification of P-gp then required its solubilisation with 2% (w/v) OG to remove it from the membrane. P-gp was then purified in the presence of E. coli lipids and cholesterol using Ni-NTA affinity chromatography. The detergent was removed using BioBeads forcing reconstitution of P-gp into proteoliposomes. The efficiency of this reconstitution could be monitored using a sucrose density gradient. A successful reconstitution showed both protein and lipid in the same layer of the sucrose gradient. ATPase activity was measured via a colorimetric assay based on the amount of inorganic phosphate released during hydrolysis of ATP to ADP+Pi (Chifflet et al., 1988). Vmax and Km were calculated using the Michaelis-Menten equation as 2.6 μmol Pi/min/mg P-gp (n=4, SEM 0.15) and 0.6 mM ATP (n=4, SEM 0.04) in the presence of 30 μM nicardipine and basal values of 0.5 μmol Pi/min/mg P-gp (n=4, SEM 0.06) and 0.4 mM ATP (n=4, SEM 0.07). In addition the EC50 of nicardipine was determined to be 24.2 μM. These methods were well established by Taylor et al. before their use in this investigation.

### EXAMPLE 2

### Freeze Drying P-gp Proteoliposomes with Trehalose

### 2.1 Introduction.

### 2.1.1 Survival in the Dry State and Trehalose.

Anhydrobiosis in organisms is commonly associated with high levels of disaccharides and in particular trehalose (Crowe, L. M., 2002). Trehalose or α-D-glucopyranosyl (1-1) α-D-glucopyranose is a non-reducing disaccharide which adopts either an anhydrous or a dihydrate form. The chemical structure of anhydrous trehalose is as follows:

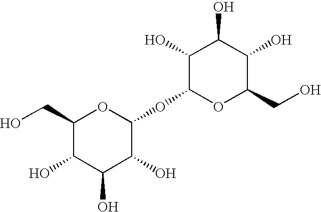

### 2.1.2 Trehalose as a Lyoprotectant in Freeze Drying.

The use of trehalose as a lyoprotectant in the dehydration, desiccation and lyophilisation of membranes and labile proteins has been well established (reviewed by Crowe J. H., et al 1992). Evidence for the role of trehalose in both the water replacement hypothesis and vitrification hypothesis exists and both theories are unlikely to be mutually exclusive. Trehalose displays both a high glass transition temperature in the amorphous state and the ability to form hydrogen bonds in an anhydrous environment (Crowe J. H. et al., 1984, Liao Y., et al. 2002, Dean Allison S., et al. 1999). The uptake of small amounts of water appears not to affect the glass transition temperature of trehalose due to the formation of trehalose dihydrate, thus protecting the remainder of the product from moisture (Crowe J. H., et al., 1998).

### 2.2 Methods.

2.2.1 Replacement of Glycerol with Trehalose.

Initial experiments showed that the glycerol content of the buffers used in purification of P-gp rendered them unsuitable for freeze drying. As is described above trehalose is common in many anhydrobiotic organisms and has been widely used as a lyoprotectant excipient in many lyophilised systems. Successful freeze drying of P-gp proteoliposomes required the removal of glycerol from the buffer whilst still retaining the cryo-protective properties of the buffer. Trehalose was therefore seen as an ideal candidate to replace glycerol in the preparation of P-gp proteoliposomes for freeze drying as it possess both cryo- and lyoprotective properties (Xie, G. and Timasheff, S. 1997).

The purification was carried out un-modified with buffers as described until wash 5 and elution. The composition of these buffers was changed to allow elution and reconstitution of P-gp into proteoliposomes in the presence of trehalose and absence of glycerol e.g. 20 mM Tris, 150 mM NaCl, 1.5 mM MgCl₂, 1.25% (w/v) OG and 0.1% (w/v) lipids/cholesterol mix, 20% (w/v) trehalose.

### 2.2.2 Does Trehalose Affect P-gp ATPase Activity or Potency of Nicardipine?

In order for trehalose to be a viable lyoprotectant it was necessary to demonstrate that there were no pharmacological changes to ATPase activity. ATPase activity was assayed in glycerol purified P-gp over a range of trehalose concentrations. The highest concentration was similar to that found in the reaction volume of an ATPase assay when a 20% (w/v) buffer was used (10 mM-300 mM). Trehalose was dissolved in ATPase buffer and added to produce the correct concentration of trehalose in the 50 μl well volume. Both varying ATP and varying drug assays were performed.

Additionally, ATPase activity was measured in P-gp purified in trehalose to confirm that the novel buffer used did not affect P-gp.

### 2.2.3 Establishment of a Freeze Drying Cycle.

All freeze drying was carried out using a CHRIST Alpha 2-4 (MartinChrist, Germany) with the condenser temperature set to −80° C. Initial experiments were performed with product in micro-centrifuge tubes. Later experiments were performed using 2 ml glass freeze drying vials and freeze drying injection stoppers in conjunction with a stoppering system. This permitted vials to be sealed under vacuum, thus avoiding moisture uptake from the atmosphere when the chamber pressure was raised to atmospheric. In all experiments the shelf was pre-cooled to −80° C. to avoid melting of products whilst the chamber was being evacuated to the required pressure.

The rate and extent to which a product is dried through lyophilisation is dependent on several factors. Whilst it was not possible to control some of these factors due to the nature of equipment and practicalities of storing and transporting samples, it was however important to characterise different drying conditions. Under direct control were the chamber pressure and the length of drying time. Various pressures and drying times were used in initial investigation of freeze drying P-gp proteoliposomes.

TABLE 2
Drying times and pressures used in optimisation of a drying cycle.
Drying times and pressures

```
0.0005 mBar 3 hrs: 0.07 mBar 17 hrs
  0.12 mBar 4 hrs: 0.011 mBar 2 hrs
  0.011 mBar 6 hrs
  0.011 mBar 24 hrs
  0.011 mBar 72 hrs
```

However, after optimisation the following times and pressures were used to investigate the effect of residual water content on freeze dried P-gp proteoliposomes.

TABLE 3
Description of drying cycles.

| Cycle number | Duration | Chamber Pressure |
|---|---|---|
| Cycle 1 | 6 hours | 0.011 mBar |
| Cycle 2 | 24 hours | 0.011 mBar |
| Cycle 3 | 48 hours | 0.011 mBar |
| Cycle 4 | 72 hours | 0.011 mBar |
| Cycle 5 | 144 hours | 0.011 mBar |

### 2.2.4 Determination of Residual Water Content by Karl Fischer Titration.

Residual water content of freeze dried P-gp proteoliposomes was determined using an AF7 Coulometric Karl Fischer (QCL Ltd., UK). Residual water was expressed as a percentage of the mass of the dried product. Karl Fischer titration determined water content via the 1:1 reaction between iodine and water as detailed in Equation 3.

I₂+H₂0+SO₂→2HI+H₂0₄

Equation 3: Chemical equation describing Karl Fischer titration. This reaction took place with methanol as a solvent. Iodine is generated coulometrically and from this the amount of water in mg is determined. Residual water was calculated as a percentage of the total weight of the dry product.

### 2.2.5 Assessment of ATPase Activity After Freeze Drying.

In order for freeze drying to be considered successful P-gp must have at least retained ATPase activity on rehydration immediately after freeze drying. On completion of the freeze drying cycle the solid product cake was rehydrated with distilled water. The product vial was vortexed to ensure the dried product was completely dissolved and rehydrated. ATPase activity was then assayed as previously described.

However, the successful stabilisation of P-gp proteoliposomes required ATPase activity to be retained for longer than currently possible at given temperatures. For example, P-gp proteoliposomes require storage at −80° C. in order to preserve ATPase activity over a period of time (FIG. 7, unpublished data, Rothnie A.).

P-gp proteoliposomes were freeze dried, sealed and stored at a range of temperatures (4° C., 20° C. and 37° C.) and ATPase activity was assessed at intervals up to 150 days.

### 2.3 Results and Discussion.

### 2.3.1 Glycerol is Unsuitable for Freeze Drying of P-gp Proteoliposomes.

The conventional purification of P-gp takes place in a buffer with 20% (v/v) glycerol. In order to investigate the stabilisation of P-gp it was necessary to establish whether or not any modification of the system was required. P-gp proteoliposomes were freeze dried according to Cycle 4 described above.

Following freeze drying liquid glycerol remained in the vial with no cake structure commonly associated with freeze dried products visible. It can be seen that following the complete loss of ATPase activity glycerol was not suitable for freeze drying P-gp proteoliposomes. See FIG. 8.

### 2.3.2 Trehalose can Substitute for Glycerol During Purification.

As described above it was necessary for purified P-gp to be eluted and incorporated into proteoliposomes in a glycerol free buffer. In order to maintain the protective properties of the buffer 20% (v/v) glycerol was substituted for 20% (w/v) trehalose in wash 5 and elution buffers (Table 1). FIG. 9 shows fractions taken during purification where glycerol was substituted for trehalose visualised on SDS-PAGE. It can be seen, as in FIG. 2, that impurities are removed during the washes and single bands corresponding to P-gp are seen in the elutions.

The buffers used in lanes 1-6 contained 20% (v/v) glycerol pH 8, lanes 7-11 contained 20% (w/v) trehalose pH 6.8.

It was also necessary to confirm that good yields of P-gp could be obtained when trehalose was used during final purification and elution. As described previously protein yield was measured by densitometric analysis of SDS-PAGE stained with Coomassie Blue. It can be seen below (see FIG. 10) that the size of the bands for P-gp eluted in glycerol and trehalose are the same, showing that yield of pure protein was unaffected by modification of the elution buffer. P-gp purified in glycerol buffer gave 115 μg (SEM 41.7, n=4) from 50 mg total membrane protein and trehalose purified P-gp yield was 108 μg (SEM 28.5, n=4).

2.3.3 Trehalose does not Affect ATPase Activity of P-gp Eluted in a Glycerol Buffer.

Before trehalose could be considered for use with P-gp it had to be established that trehalose as an excipient did not interfere with ATPase activity or drug binding. ATPase activity was measured over a range of trehalose concentrations against varying ATP and nicardipine concentrations. The highest concentration of trehalose used was 300 mM as this was calculated to be close to the final concentration of trehalose in the 50 μl reaction volume during ATPase assay when 20% (w/v) trehalose was used in the elution buffer. It can be seen that addition of trehalose to glycerol purified P-gp did not affect ATPase activity (FIGS. 11A and B) or potency of nicardipine to stimulated ATPase activity (FIG. 12). Comparison between results obtained in the presence and absence of trehalose showed that p=0.6551 and p=0.7549 (two-way ANOVA) for nicardipine stimulated ATPase activity and nicardipine binding respectively.

Table 4 below shows Vmax (μmol/min/mg) and Km (mM) data for each concentration of trehalose used as well as in the absence of trehalose. Both nicardipine stimulated and basal mean data are shown with the SEM for each value from four independent purifications. The addition of trehalose does not affect the hydrolysis of ATP by P-gp. The lack of trehalose mediated inhibition or stimulation rendered it a viable candidate for continued investigation into the lyoprotection of P-gp proteoliposomes.

TABLE 4
Values obtained from ATPase assays of glycerol purified P-gp in
the presence and absence of various trehalose concentrations.
Nicardipine simulated activities were measured at 30 μM
nicardipine. Both nicardipine stimulated and basal Vmax's
and Km's shown are means of n = 4 independent
purifications, values in parenthesis are SEM.

| Trehalose | Nicardipine stimulated | | Basal | |
|---|---|---|---|---|
| concentration | Vmax | | Vmax | |
| (mM) | (μmol/min/mg) | Km (mM) | (μmol/min/mg) | Km (mM) |
| 0 | 4.3 (1.7) | 1.3 (0.98) | 1.1 (1.1) | 2.7 (4.1) |
| 10 | 4.7 (1.6) | 1.4 (0.93) | 0.86 (0.67) | 1.6 (2.3) |
| 30 | 5.6 (2.3) | 1.6 (1.2) | 0.63 (0.4) | 1.1 (1.5) |
| 100 | 4.9 (2.1) | 1.6 (1.2) | 1.0 (0.86) | 2.0 (2.9) |
| 300 | 3.7 (1.3) | 1.2 (0.88) | 0.7 (0.54) | 1.7 (2.3) |

### 2.3.4 P-gp Eluted in the Absence of Glycerol and Presence of Trehalose.

Once established that trehalose did not directly alter ATPase activity or drug binding, it was necessary to show that ATPase activity is similar in P-gp eluted in glycerol and trehalose. The nicardipine stimulated Vmax's and Km's of P-gp purified in both glycerol and trehalose are compared in the Table 5.

TABLE 5
Comparison of mean Vmax and Km in presence of nicardipine
from P-gp purified in glycerol and trehalose. Results are from 4
independent purifications.

| | | Standard | | Standard |
|---|---|---|---|---|
| Buffer | Vmax | error of the | Km (mM | error of the |
| composition | (μmol/min/mg) | mean | ATP) | mean |
| Glycerol | 2.60 | 0.16 | 0.60 | 0.04 |
| Trehalose | 2.58 | 0.83 | 0.43 | 0.23 |

It was found that P-gp could still be purified with good ATPase activity when glycerol was replaced with trehalose. ATPase activity was maintained both before and after freezing proteoliposomes at −80° C. indicating that 20% (w/v) trehalose had cryo-protective properties similar to 20% (v/v) glycerol.

### 2.3.5 Establishment of a Freeze Drying Cycle.

Due to the small volume of samples to be freeze dried (400 μl) it was necessary to pre-cool the shelf used during freeze drying by incubation at −80° C. for at least 1 hour before lyophilisation of samples. This served the dual purpose of keeping samples frozen whilst the vacuum was pulled and maintaining the temperature of samples safely below melting during primary drying.

Primary drying was identified as ending after 3-4 hours when the product temperature rose dramatically to meet that of the shelf (FIG. 13). This was an indication of all ice having sublimated and only bound water remaining.

Without the facility to control the temperature of the shelf directly, final shelf temperature was dictated by ambient room temperature. Whilst every effort was made to ensure that room temperature varied as little as possible, it was inevitable that conditions were not identical for each freeze drying. As a result of this the time periods allowed for secondary drying to occur were large in an attempt to minimise different environmental conditions. It was noted that chamber pressures in the literature were significantly higher than 0.011 mBar, typically 0.1-0.2 mBar (Liao Y. et al. 2002, Dean Allison S. et al. 1999 and Carpenter J. et al. 1993). However, chamber pressure during secondary drying is lowered to encourage desorption of bound water e.g. 0.01 mBar (van Winden E. et al. 1999) and given the short primary drying time identified in Cycle 4 it was decided that 0.011 mBar would be used throughout in a one-step cycle.

### 2.3.6 Determination of Residual Water Content.

To accurately determine the extent to which water was removed during the freeze drying cycle, minimising environmental influence, samples were sealed before the chamber pressure was returned to atmospheric. Residual water was calculated as a percentage of the dry mass of product and after 72 hours at 0.011 mBar was determined by Karl Fischer titration as 4.2% (SEM 0.35, n=4 independent experiments); see FIG. 14.

### 2.3.7 ATPase Activity is Retained on Rehydration of Freeze Dried P-gp Proteoliposomes.

Freeze dried P-gp proteoliposomes were rehydrated immediately after the end of the cycle and ATPase assay was measured as previously described. Rehydration was with 330 μl room temperature distilled water. This volume was used as the mean dry cake weight was 66.7 mg (SEM 1.25, n=4) and in order to accurately measure and compare ATPase activity the concentration of P-gp used in pre and post freeze drying assays must be as close as possible. P-gp retains 83.0% (SEM 19.7% n=4) of its pre-freeze dried maximal nicardipine stimulated ATPase activity. The Km for pre and post freeze dried samples was 0.66 mM ATP and 0.73 mM ATP (SEM 0.41, n=4) respectively, showing that the affinity for ATP was unaffected (FIG. 15).

### 2.3.8 ATPase Activity is Retained After Prolonged Storage of Freeze Dried P-gp Proteoliposomes.

As stated in the methods (2.2.5) preservation of ATPase activity over prolonged periods of time would constitute successful stabilisation of P-gp proteoliposomes. The results of the storage tests revealed that P-gp did indeed retain ATPase activity for several months. Different storage temperatures did have an effect on the extent to which activity was preserved in the order 4° C.>20° C.>37° C. with the difference between 4° C. and 20° C. being the smallest. In addition to the Vmax being preserved the Km remained unchanged over time apart from two anomalous results at 12 days. This indicates that the characteristics of the ATP hydrolysis where unaffected by freeze drying and storage.

The data in FIGS. 16A and B shows that trehalose is therefore capable of acting as a lyoprotectant for proteoliposomes over a prolonged period of time.

### 2.4 Summary of Results.

The results presented above show that trehalose was a viable candidate as a lyoprotectant. Trehalose was seen to inhibit the ATPase activity of the plasma membrane H⁺-ATPase at high concentrations (0.6 M to 0.8M) at 20° C. due to the viscosity of the solution hindering protein conformational change and ATP diffusion. However, at 40° C. the reduction in Vmax was negligible (Sampedro J. et al. 2002). Trehalose in concentrations up to 300 mM does not affect nicardipine stimulated or basal ATP hydrolysis p=0.6551 and p=0.9422. The potency of nicardipine to stimulate ATPase activity is similarly unaffected with an EC50 of 19.2 μM in the presence of trehalose compared with 24.2 μM in the absence of trehalose, with no significant difference between the curves (p=0.7549). It was also demonstrated that trehalose can substitute for glycerol in the final part of the purification process. No loss of ATPase activity was measured when 20% (w/v) trehalose was used instead of 20% glycerol (v/v) in the buffer used to elute P-gp from the column. Yields of protein were also comparable as measured by densitometric analysis of SDS-PAGE with a BSA standard curve (FIG. 10).

Whilst buffers containing glycerol proved totally unsuitable for freeze drying, trehalose allowed drying of samples to ˜4% residual moisture over a 72 hour period even without heating of the shelf above ambient temperatures. Importantly, on rehydration of freeze dried P-gp proteoliposomes over 80% ATPase activity was recovered immediately and activity persisted at up to 60% for 150 days at 4° C. and 20° C., whilst at 37° C. around 40% activity was retained over the same time.

### EXAMPLE 3

### Freeze Drying P-gp Proteoliposomes with Maltose

### 3.1 Introduction.

### 3.1.1 Maltose in Nature.

Maltose or α-D-glucopyranosyl (1-4) α-D-glucopyranose is a naturally occurring disaccharide formed by the hydrolysis of starch. Due to the (1-4) glycosidic link between the two glucose monomers, maltose unlike trehalose is a reducing sugar. Maltose forms a monohydrate but also exists in anhydrous form. The chemical structure of anhydrous maltose is as follows:

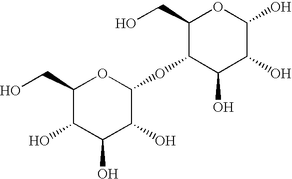

### 3.1.2 Maltose as a Lyoprotectant.

Whilst not widely used in freeze drying studies, maltose has been shown to be second only to trehalose in protecting a yeast plasma membrane protein (Sampedro J. G. et al. 2002). Use has been made of maltose's ability to form maltooligosaccharides to investigate the effect of increasing molecular weight on protection of protein secondary structure. It was shown that whilst maltose was more effective than glucose (the component molecules of maltose), structure stabilisation was reduced with the addition of units above the disaccharide (Izutsu K. et al., 2004). The same trend was observed when the effect of increasing maltooligosaccharides size on the stabilisation of freeze dried liposomes was investigated (Suzuki T. et al. 1996, Ozaki K. and Hayashi M. 1997). That is, that the disaccharide form is superior to the larger sugars.

### 3.2 Methods.

3.2.1 Replacement of Glycerol with Maltose.

The unsuitability of glycerol for freeze drying required its substitution to allow purification and freeze drying of P-gp proteoliposomes. It has been shown that trehalose displays both cryo- and lyoprotective qualities and allowed for the successful purification of P-gp. In order to investigate the comparative abilities of trehalose and maltose it was necessary that P-gp could be purified successfully with a buffer containing maltose. The final wash and elution was thus altered to 20 mM Tris, 150 mM NaCl, 1.5 mM MgCl₂, 1.25% (w/v) OG and 0.1% (w/v) lipids/cholesterol mix, 20% (w/v) maltose.

### 3.2.2 Does Maltose Affect P-gp ATPase Activity or Potency of Nicardipine?

Despite the chemical similarity of maltose and trehalose, it is possible that the structural difference may have lead to maltose having some property not exhibited by trehalose regarding the hydrolysis of ATP and binding of nicardipine. In order to demonstrate that any differences observed were the result of freeze drying in the presence of a different disaccharide and not the presence of the disaccharide per se, the same modified ATPase assays were performed on glycerol purified protein over a range of maltose concentrations.

A comparison was also made between the glycerol purified protein and that purified in the maltose buffer.

### 3.2.3 Freeze Drying.

The optimisation process was not repeated, with Cycle 4 being used due to the similarity of the products. Use of freeze drying vials which allowed for sealing under vacuum and cooling of the shelf was identical to experiments performed on trehalose based products.

### 3.2.4 Determination of Residual Moisture.

Residual water content of freeze dried products was performed by coulometric Karl Fischer analysis as described previously and expressed as a percentage of dry product weight.

### 3.2.5 Assessment of ATPase Activity After Freeze Drying.

Following rehydration of P-gp proteoliposomes the ATPase activity was measured as described earlier. Activity was assessed following storage at 4° C., 20° C. and 37° C. at intervals up to 30 days.

### 3.3 Results and Discussion.

### 3.3.1 Maltose can Substitute for Glycerol During Purification.

The replacement of 20% (v/v) glycerol with 20% (w/v) maltose did not affect the purification of P-gp. As can be seen in FIG. 17, P-gp was successfully purified with the removal impurities and only a band corresponding to the position of P-gp when visualised on SDS-PAGE.

The buffers used in lanes 1-6 contained 20% (v/v) glycerol pH 8, lanes 7-11 contained 20% (w/v) maltose pH 6.8.

Yields of protein purified in the maltose buffer were similar to those in both the glycerol and trehalose buffers (FIG. 18).

3.3.2 Maltose does not Affect ATPase Activity of P-gp Eluted in a Glycerol Buffer.

As with the trehalose buffer, it was important to establish there was no direct effect of maltose on the ATPase activity of P-gp or the potency of nicardipine to stimulate ATPase activity. The same modified ATPase assay was used as described in 2.2.2 except trehalose was replaced with maltose. There was no significant affect on the nicardipine stimulated Vmax over the range of concentrations tested with p=0.4215 (FIGS. 19A and B). As can be seen from Table 6, the Km and basal Vmax and Km also remained unchanged in the presence of maltose. Thus, maltose could be explored as a lyoprotectant with the knowledge that ATP hydrolysis by P-gp was unaffected. Given that trehalose and maltose are very similar molecules and no effect was observed with increasing trehalose concentration, it would have seemed unlikely that maltose would have exerted any effect.

TABLE 6
Values obtained from ATPase assays of glycerol purified
P-gp in the presence and absence of various maltose
concentrations. Nicardipine simulated activities were
measured at 30 μM nicardipine, Vmax's and Km's
shown are means of n = 4 independent purifications,
values in parenthesis are SEM.

| Maltose | Nicardipine stimulated | | Basal | |
|---|---|---|---|---|
| concentration | Vmax | | Vmax | |
| (mM) | (μmol/min/mg) | Km (mM) | (μmol/min/mg) | Km (mM) |
| 0 | 1.2 (0.3) | 1.2 (0.7) | 0.1 (0.1) | 0.3 (0.7) |
| 10 | 1.7 (1.1) | 2.5 (2.6) | 0.1 (0.4) | 0.4 (9.8) |
| 30 | 1.1 (0.6) | 1.3 (1.3) | 0.1 (0.1) | 0.4 (1.0) |
| 100 | 1.0 (0.3) | 1.3 (0.8) | 0.1 (0.6) | 0.1 (0.3) |
| 300 | 0.9 (0.4) | 1.5 (1.1) | 0.1 (0.2) | 0.9 (2.8) |

Again, maltose had no effect on the interaction between P-gp and nicardipine with p=0.5249 (FIG. 20). Therefore maltose itself, like trehalose, did not alter the ATPase activity of P-gp purified in glycerol.

### 3.3.3 P-gp Eluted in the Absence of Glycerol and Presence of Maltose.

In order to proceed with maltose as a possible lyoprotectant, it was necessary to determine if P-gp could be eluted in maltose without loss of ATPase activity. There was no real loss of ATPase activity in the presence of 30 μM nicardipine in P-gp eluted in 20% (w/v) maltose buffer. It is important to note that though the activities in Table 6 are lower than those reported previously, this is not an indication of maltose being unsuitable for use in purification as the values for conventionally glycerol purified P-gp are also low. This is a result of variation in cell culture conditions and subsequent protein expression levels and localisation.

TABLE 7
Comparison of mean Vmax and Kd in presence of 30 μM
nicardipine from P-gp purified in glycerol and maltose.
Results are from 4 independent purifications.

| | | Standard | | Standard |
|---|---|---|---|---|
| Buffer | Vmax | error of the | Kd | error of the |
| composition | (μmol/min/mg) | mean | (mM ATP) | mean |
| Glycerol | 0.60 | 0.16 | 0.54 | 0.12 |
| Maltose | 0.50 | 0.14 | 0.60 | 0.11 |

Maltose could therefore be investigated as a possible protectant during freeze drying as it allowed for the purification and freezing of P-gp whilst retaining ATPase activity.

### 3.3.4 Freeze Drying and Determination of Residual Water.

An identical cycle was used to freeze dry P-gp eluted in the maltose as was used with trehalose eluted P-gp, e.g. cooling the shelf to −80° C., then evacuating the chamber to 0.011 mBar for 72 hours followed by sealing freeze drying vials under vacuum.

Residual water content was again determined by coulometric Karl Fischer titration. Maltose eluted samples gave extremely similar levels of residual moisture after freeze drying as trehalose samples. This is unsurprising as maltose and trehalose are chemically identical, though maltose forms a monohydrate compared to trehalose forming a dihydrate. Residual water in freeze dried maltose eluted P-gp was found to be 4.8% (SEM 0.25, n=4). This was also reflected in the mass of the dried product. The dry product in trehalose eluted and freeze dried P-gp was 66.7 mg (SEM 1.25, n=4), whereas with maltose the mass rose to 66.9 mg (SEM 1.35, n=4).

### 3.3.5 ATPase Activity is Retained on Rehydration of Freeze Dried P-gp Proteoliposomes.

On rehydration following freeze drying P-gp proteoliposomes displayed nicardipine stimulated ATPase activity. However, whilst those purified in trehalose showed high levels of recovered ATPase activity (see 2.3.7) those eluted in maltose showed slightly lower levels of recovered activity with 69.9% (SEM 21.6% n=4). Recovered activity was defined as the post-freeze dried nicardipine stimulated Vmax divided by the pre-freeze dried nicardipine stimulated Vmax. Whilst the Vmax was shown to be reduced following freeze drying, affinity for ATP remained unaffected. Km pre-freeze drying was 0.91 mM ATP and post freeze drying 1.2 mM ATP (SEM 0.83, n=4). See FIG. 21.

### 3.3.6 ATPase Activity is Rapidly Reduced After Prolonged Storage of Freeze Dried P-gp Proteoliposomes.

The ability of maltose to preserve ATPase activity after freeze drying was tested over a range of temperatures and time as described earlier. It was observed that ATPase activity in maltose purified P-gp decreased relatively rapidly compared to the P-gp eluted in trehalose. FIG. 22A shows that activity fell off immediately in samples stored above 4° C.; however, the effect was less at 20° C. than at 37° C. After only 12 days, samples stored at 4° C. also began to decline and at 30 days samples stored at all temperatures had less than 50% of their pre-freeze dried nicardipine stimulated Vmax.

By direct comparison with storage data from trehalose purified P-gp proteoliposomes over a similar time period (FIG. 22B), it can be seen that maltose is far less effective at preserving ATPase activity in the dried state. P-gp purified in maltose not only retains less ATPase activity following freeze drying initially (3.3.5), but activity also declines more rapidly at all temperatures tested than the trehalose equivalent.

Whilst the Vmax dropped rapidly over the time period measured, the Km did change. This suggests whilst the amount of ATP hydrolysed over time decreased, the affinity of P-gp for ATP was not altered (FIG. 23).

These data, taken together, show that whilst maltose can protect P-gp proteoliposomes from immediate freeze drying damage, it is not suitable for use over any length of time at any of the temperatures tested.

### 3.4 Summary of Results.

It has been shown above that maltose does not effect P-gp ATPase activity or nicardipine potency per se with values of p=0.4215 and p=0.5249 respectively. This shows that there is no significant difference between activity in the presence and absence of a range of concentrations of maltose. In addition to this, a maltose based buffer can be used to elute P-gp in place of a glycerol based buffer without loss of purity, yield or ATPase activity.

Interestingly, freeze dried maltose formulations exhibited slightly higher residual water measurements than trehalose; 4.8% (SEM 0.25, n=4) as opposed to 4.2% (SEM 0.35, n=4). Whilst the difference is not large, it is the opposite of what would be expected had the remaining water been held in crystalline disaccharide hydrates. As stated earlier, maltose forms a monohydrate, that is, one molecule of water per molecule of maltose; trehalose however forms a di-hydrate. This suggests that whilst ˜4% seems high for some freeze dried formulations with typically ˜1% residual moisture, the remaining water in the proteoliposome system is not “free” water but rather bound water not removed during the desorption stage of the cycle.

The use of maltose in the formulation also affords some degree of lyoprotection with 69.9% of pre freeze dried ATPase activity being recovered on re-hydration following freeze drying compared to 83.0% in trehalose. However, a major difference between trehalose and maltose was observed when ATPase activity was assessed over time and a range of temperatures. Maltose was unable to preserve P-gp ATPase activity for more than 12 days, and even then only at 4° C. At higher temperatures the loss of recovered activity was rapid. The cause of this lack of protection and the stark contrast with that afforded by trehalose will need to be explored. The large difference in lyoprotection is of interest due to the fact is that the disaccharides are structurally so similar.

1. A method for producing purified P-glycoprotein (P-gp) which comprises:
i) solubilising a mixture of P-gp and other proteins obtained from cell membranes;
ii) removing insoluble material by centrifugation;
iii) placing the mixture of P-gp and other proteins in a purification column; and
iv) running through the purification column at least one wash buffer so as to remove unwanted proteins, and then at least one elution buffer so as to recover the P-gp; wherein the elution buffer used in the final elution step of the method contains a disaccharide or a mixture of disaccharides.

2. A method as claimed in claim 1, wherein a series of wash buffers and elution buffers is used during the method.

3. A method as claimed in claim 1, wherein one or more of the wash buffers contains a disaccharide or a mixture of disaccharides.

4. A method as claimed in claim 1, wherein the disaccharide is trehalose.

5. Purified P-gp produced by the method of claim 1.

6. P-gp proteoliposomes reconstituted from the purified P-gp produced by the method of claim 1.

7. A screening system for drugs which comprises providing P-gp or P-gp proteoliposomes produced by the method of claim 1.

8. A method as claimed in claim 1, wherein the wash buffer used in the final wash step of the method contains a disaccharide or a mixture of disaccharides.
